SEQUENCE LISTING (9 sequences)

SEQ ID NO: 1 - DNA, 21 bp, Artificial
  PCR
ccattcgaacgtctgccctat

SEQ ID NO: 2 - DNA, 18 bp, Artificial
  PCR
tcacccgtggtcaccatg

SEQ ID NO: 3 - DNA, 27 bp, Artificial
  PCR
actttcgatggtagtcgccgtgcctta

SEQ ID NO: 4 - DNA, 19 bp, Artificial
  PCR
tggccagcttcgagaaaga

SEQ ID NO: 5 - DNA, 21 bp, Artificial
  PCR
gcgctgtgacttgtggtctgt

SEQ ID NO: 6 - DNA, 30 bp, Artificial
  PCR
agaagttaaacatgctcagcattgatcata

SEQ ID NO: 7 - DNA, 23 bp, Artificial
  PCR
actcatccaagtgatggctgaac

SEQ ID NO: 8 - DNA, 24 bp, Artificial
  PCR
ccttgaaacagcatctgactcctt

SEQ ID NO: 9 - DNA, 27 bp, Artificial
  PCR
tcgccagcagctaaaacagggaagcta

(19) United States
(12) Patent Application Publication
(10) Pub. No.: US 2011/0257256 A1
(43) Pub. Date: Oct. 20, 2011

(54) CANNABINOIDS FOR USE IN TREATING OR PREVENTING COGNITIVE IMPAIRMENT AND DEMENTIA

(75) Inventors: Dietmar Fuchs, Innsbruck (AT); Marcel Jenny, Innsbruck (AT); Eberhard Pirich, Wien (AT)

(73) Assignee: BIONORIA RESEARCH GMBH, Innsbruck (AT)

(21) Appl. No.: 13/056,713

(22) PCT Filed: Jul. 31, 2009
(86) PCT No.: PCT/EP2009/005701

§ 371 Date: Jun. 22, 2011

Related U.S. Application Data
(62) Division of application No. 61/085,171, filed on Jul. 31, 2008.

(51) Int. Cl. A61K 31/352; A61P 25/16; A61P 25/28; A61K 31/05

(52) U.S. Cl. 514/454; 514/733

(57) ABSTRACT

The present invention is directed to the use of at least one cannabinoid in the manufacture of a medicament for use in treating or preventing or in the manufacture of a dietary supplement for preventing a disease or condition benefiting from a reduced activity of the enzyme indoleamine 2,3-dioxygenase (IDO). The disease or condition to be treated or prevented is preferably selected from cognitive impairment or any kind of dementia.

The present invention relates to the use of at least one cannabinoid in the manufacture of a medicament for use in treating or preventing a disease or condition benefiting from a reduced activity of the enzyme indoleamine 2,3-dioxygenase (IDO). The present invention also relates to the use of at least one cannabinoid in the manufacture of a dietary supplement for preventing such a disease or condition. Preferably, the diseases or conditions to be treated or prevented are selected from cognitive impairment and dementia.

Δ9-Tetrahydrocannabinol (THC) is the main psychoactive cannabinoid produced by Cannabis sativa (L.) or Cannabis indica (Lam.) which is well characterized for its biological activity and potential therapeutic application in a broad spectrum of diseases. The semi-synthetic form of THC, Dronabinol (or Marinol™), is approved in the U.S. for the treatment of patients with cancer and AIDS to achieve medical benefit by increasing appetite, decreasing nausea and vomiting associated with chemotherapy and, e.g. blocking the spread of Herpes simplex viruses. Cannabis species produce more than 60 cannabinoids, the most abundant thereof is the non-psychotropic cannabinoid cannabidiol (CBD), which is reported to exert analgesic, antioxidant, anti-inflammatory, and immunomodulatory effects but bears also the capacity to decrease several adverse effects of THC such as sedation, tachycardia and anxiety. The discovery of specific cannabinoid receptors, especially on cells of the immune system, has generated growing interest in evaluating the potential of cannabinoids as anti-inflammatory and immunomodulatory agents.

Cannabinoids exhibit their biological effects by mimicking the endogenous ligands anandamide or 2-arachidonoylglycerol which bind and activate specific G protein-coupled receptors termed cannabinoid (CB) receptors 1 and 2 and are synthesized on demand in response to increasing levels of intracellular calcium. Whereas CB1 receptors are mainly found in the mammalian brain and at much lower concentrations in peripheral tissues and cells, CB2 receptors are predominantly expressed on cells of the immune system, but just recently were reported to be also present in brain stem neurons. In human peripheral blood mononuclear cells (PBMC), CB2- and at much lower concentrations also CB1-mRNA levels are most abundant in B cells and at lower levels also in monocytes and T cells. THC is reported to activate both CB1 and CB2 receptors with Kᵢ values in the low nanomolar concentration range. However, because synthetic agonists such as HU-210, CP55940 or Win55212 exhibit higher CB1/CB2 efficacy in comparison to THC, this cannabinoid is considered to act as a partial agonist of CB1 and CB2 receptors. In contrast, CBD displays low affinity for these receptors (in the micromolar range) but nevertheless CBD has been shown to antagonize CB1/CB2 agonists with K_{B} values in the low nanomolar range and thus is regarded as an inverse agonist. The expression levels of CB1 and CB2 on immunocompetent cells was reported to be variably regulated in marijuana users and in vitro by various stimuli that induce immune activation such as phytohemagglutinin (PHA), lipopolysaccharide (LPS), phorbol myristate acetate (PMA), cytokines or mitogenic antibodies.

THC was found to exhibit marked immunosuppressive effects on macrophages, natural killer (NK) cell activity and T lymphocytes. These effects include suppression of mitogen-stimulated proliferation, interleukin (IL)-2 production, T cell-dependent antibody responses and inhibition of macrophage secretion of the proinflammatory cytokine tumor necrosis factor-α (TNF-α). Furthermore, THC was also reported to regulate the Th1-/Th2-type cytokine balance in activated human T cells polarizing the immune response towards a Th2 phenotype. Inhibition of Th1-type cytokines and/or propagation of a Th2-type response are considered to be beneficial in various inflammatory diseases, suggesting cannabinoids as promising agents in the treatment of such disorders.

Stimulation of PBMC with mitogens like PHA induces production of Th1-type cytokine interferon-γ (IFN-γ) which in turn activates in macrophages the enzyme indoleamine 2,3-dioxygenase (IDO) that converts tryptophan into N-formylkynurenine, which is subsequently deformylated to kynurenine. In parallel to tryptophan degradation, neopterin concentrations increase in mitogen stimulated PBMC representing another marker for the activation of the T cell-macrophage axis in humans. Likewise, in diseases which are associated with inflammation and immune activation, accelerated tryptophan degradation manifests in decreased serum tryptophan concentrations and increased kynurenine to tryptophan ratio (kyn/trp). The decreased availability of tryptophan in such conditions was found to be associated with reduced quality of life and an increased risk of depression, e.g., in patients with cancer or undergoing treatment with pro-inflammatory cytokines.

The objective of the current study was to evaluate the effects of cannabinoids THC and CBD on mitogen-induced degradation of tryptophan and formation of neopterin using freshly isolated human PBMC. Additionally, the influence of these cannabinoids on LPS-induced tryptophan metabolism was investigated in the myelomonocytic THP-1 cell line.

The present inventors surprisingly found that cannabinoids and in particular THC and CBD have a pronounced effect on the enzyme indoleamine 2,3-dioxygenase (IDO) as well as on the tryptophan metabolism and the serotonergic system.

Accordingly, the present invention is directed to the use of at least one cannabinoid in the manufacture of a medicament for use in treating or preventing a disease or condition benefiting from a reduced activity of the enzyme indoleamine 2,3-dioxygenase (IDO).

Under a further aspect, the present invention relates to the use of at least one cannabinoid in the manufacture of a dietary supplement for preventing a disease or condition benefiting from a reduced activity of the enzyme IDO.

In this regard, the present inventors found that cannabinoids which lead to an increased level of circulating tryptophan are especially preferred.

The at least one cannabinoid is preferably Δ9-tetrahydrocannabinol (THC, Δ9-THC, IUPAC: (6aR,10aR)-6,6,9-Trimethyl-3-pentyl-6a, 7,8,10a-tetrahydro-6H-benzo[c]chromen-1-ol, CAS: 1972-08-3) or cannabidiol (CBD, IUPAC: 2-[(1R,6R)-3-methyl-6-prop-1-en-2-yl-1-cyclohex-2-enyl]-5-pentylbenzene-1,3-diol, CAS: 13956-29-1) or a derivative thereof or a combination of THC and CBD or derivatives thereof. Derivatives are for example pharmaceutically acceptable salts, isomers, enatiomers of such compounds. Such salts are well known to a person skilled in the art. However, all other kinds of derivatives reducing the activity of the enzyme IDO may be used.

Since the present inventors surprisingly found that CBD was about two times more active as THC to suppress mitogen-induced tryptophan degradation, neopterin formation and production of interferon-gamma in stimulated human peripheral blood mononuclear cells, the use of CBD as an inhibitor or modulator of IDO is particularly preferred.

Hence, the present invention refers to a mixture of cannabinoids, wherein such mixture may have less than 10%, 5% w/w THC and/or more than 15%, 20% w/w CBD, preferably less than 2%, 1%, 0.2%, 0.1% w/w THC and/or more than 25%, 30% w/w CBD.

In a very preferred embodiment the mixture of cannabinoids is substantially free of THC or the content of THC is 0% w/w THC.

The at least one cannabinoid may be in the form of an extract prepared from at least one cannabis plant. The extract can be prepared by any method known to a person skilled in the art, for example by extraction with supercritical carbon dioxide (EP1326598) or extraction with heated gases or extraction with suitable organic or inorganic solvents, like alcohols, preferably ethanol and others.

Hence, the present invention is also directed to extracts obtainable/derivable from cannabis plants. In a very preferred embodiment the cannabis plant is Beniko, Epsilon 68, Futura 75, Felina 34, Ferimon 12, Fedora 17 (so called “Faserhanf”) due to the fact that such plants are substantially free of psychoactive THC and the main compound is CBD beside other cannabinoids. However, CBD is the main compound in such an extract obtainable from cannabis plants.

The extracts can be obtained by means of water/alcohol and other solvents based on each obtained fractions. Such methods are well known in the state of the art.

In a preferred embodiment the extracts may have less than 10%, 5% w/w THC and/or more than 15%, 20% w/w CBD, preferably less than 2%, 1%, 0.2%, 0.1% w/w THC and/or more than 25%, 30% w/w CBD.

In a very preferred embodiment the extract is substantially free of THC or the content of THC is 0% w/w THC.

Moreover, the ratio between THC:CBD within a mixture of cannabinoids or an extract may have values (w/w) of 1:2, 1:3, 1:4, 1:5, 1:10, 1:20, preferably 1:100, more preferably 1:1000.

However, the at least one cannabinoid may also be used in a substantially pure or isolated form or in a semi-synthetic or synthetic form.

Preferably, the disease or condition to be treated or prevented is selected from cognitive impairment and most preferably any kind of dementia. In particular the disease or condition is selected from the group consisting of: vascular dementia, Lewy body dementia, frontotemporal dementia, HIV-associated dementia, dementia pugilistica, corticobasal degeneration, or hereditary dementia.

However, the clinical indication dementia is preferred.

In accordance with the invention dementia shall mean a non-specific illness syndrome (set of signs and symptoms) in which affected areas of cognition may be memory, attention, language, and problem solving. It is normally required to be present for at least 6 months to be diagnosed, cognitive dysfunction which has been seen only over shorter times, particularly less than weeks, must be termed delirium. In all types of general cognitive dysfunction, higher mental functions are affected first in the process. Especially in the later stages of the condition, affected persons may be disoriented in time (not knowing what day of the week, day of the month, or even what year it is), in place (not knowing where they are), and in person (not knowing who they are or others around them). dementia, though often treatable to some degree, is usually due to causes which are progressive and incurable. Symptoms of dementia can be classified as either reversible or irreversible, depending upon the etiology of the disease. Less than 10 percent of cases of dementia are due to causes which may presently be reversed with treatment. Causes include many different specific disease processes, in the same way that symptoms of organ dysfunction such as shortness of breath, jaundice, or pain are attributable to many etiologies. However, some mental illnesses, including depression and psychosis, may also produce symptoms which must be strictly differentiated from dementia in accordance with the invention.

Moreover, the invention encompasses in a further and preferred embodiment of the invention such dementia being involved with a basic and underlying disease such Huntington, Parkinson, Alzheimer or Creutzfeldt-Jakob disease.

Preferably, the at least one cannabinoid is formulated as a pharmaceutical composition comprising in addition one or more pharmaceutically acceptable carriers or diluents.

The medicinal drugs that are manufactured with compounds or extracts in accordance with the invention can be administered orally, intramuscularly, peri-articularly, intra-articularly, intravenously, intraperotoneally, subcutaneously, or rectally. The invention pertains to processes for the manufacture of medicinal drugs that are characterized by the feature that at least one cannabinoid and/or mixture of cannabinoids and/or extracts according to the invention is/are brought into a suitable form of agent for administration together with a pharmaceutically suitable and physiologically tolerated vehicle and, optionally, further suitable active substances, additives, or ancillary substances. Suitable solid or liquid galenic forms of preparation or formulations are, for example, granulated materials, powders, sugar-coated pills, tablets, (micro)capsules, suppositories, syrups, juices, suspensions, emulsions, drops, or injectable solutions as well as preparations with a protracted release of the active substance, whereby use is made in their preparation of conventional ancillary substances, such as vehicle substances, agents that lead to the disintegration of the preparation, binders, coating agents, swelling agents, slippage promoting agents or lubricants, taste improving agents, sweeteners, and solubilizers. Mention may be made of the following as ancillary substances: magnesium carbonate, titanium dioxide, lactose, mannitol and other sugars, talcum, milk protein, gelatine, starch, cellulose and its derivatives, animal and vegetable oils such as cod-liver oil, sun flower oil, groundnut [oil] or sesame oil, polyethylene glycols), and solvents such as, for example, sterile water and monohydric or polyhydric alcohols, e.g. glycerine.

The medicinal drugs are preferably manufactured and administered in dosage units, whereby each unit contains, as the active component, a defined dose of the at least one cannabinoid and/or mixture of cannabinoids and/or extracts according to the invention. In the case of solid dosage units, such as tablets, capsules, sugar-coated pills or suppositories, this dose can amount to 1 to 1000 mg and preferably 50 to 300 mg, and in the case of injection solutions in ampoule form, this dose can amount to 0.3 to 300 mg and preferably 10 to 100 mg.

Daily doses of 20 to 1000 mg of active substance, and preferably 100 to 500 mg of active substance, are indicated for the treatment of an adult patient weighing 50 to 100 kg, e.g. 70 kg. However, higher or lower daily doses can also be applied under certain circumstances. The administration of the daily dose can take place via an administration on one single occasion in the form of an individual dosage unit or several smaller dosage units, or via the multiple administration of subdivided doses at defined intervals.

In the following, the present invention is described in more detail by way of examples. However, these examples are not intended to limit the scope of protection of the present invention in any way.

The examples also refer to several figures, the legends of which are given below:

### EXAMPLES

THC purchased from Sigmapharm (Vienna, Austria), and cannabidiol obtained from Bionorica Research (Innsbruck, Austria) were dissolved in ethanol and stored at −20° C. until use. LPS, concanavalin A (Con A) and PHA were purchased from Sigma Aldrich (Vienna, Austria), dissolved in phosphate buffered saline (PBS) and stored at −20° C. until use.

### Isolation of Human Peripheral Blood Mononuclear Cells (PBMC)

PBMC were isolated from whole blood obtained from healthy donors of whom written informed consent was obtained that their donated blood might be used for scientific purposes in case when it was not selected for transfusion. Separation of blood cells was performed using density centrifugation (Lymphoprep, Nycomed Pharma AS, Oslo, Norway). After isolation, PBMC were washed three times in phosphate buffered saline containing 0.2% 0.5 mM EDTA. Cells were maintained in RPMI 1640 supplemented with 10% heat-inactivated fetal calf serum (Biochrom, Berlin, Germany), 2 mM glutamine (Serva, Heidelberg, Germany) and 0.05 mg/ml gentamicin (Bio-Whittaker, Walkersville, Md.) in a humidified atmosphere containing 5% CO₂ for 48 h. This procedure was observed earlier to yield best reproducible results when applied for testing of anti-inflammatory effects of compounds or drugs (Winkler et al., 2007, Int. Arch. Allergy Immunol. 142, 127-132). For each of the four experiments run in duplicates, PBMC were freshly prepared.

### Stimulation of PBMC

Isolated PBMC were plated at a density of 1.5×10⁶ cells/ml in supplemented RPMI 1640 and pre-incubated for 30 min with or without THC or CBD. Consequently, the cells were stimulated or not with 10 μg/ml PHA or Con A for 48 h.

### THP-1 Cell Culture

Myelomonocytic THP-1 cells were obtained from European Collection of Cell Cultures (ECACC). The cells were plated at a density of 1×10⁶ cells/ml in supplemented RPMI 1640 and pre-incubated for 30 min with or without THC or CBD. Afterwards, cells were stimulated or not with 1 μg/ml LPS for 48 h.

### Measurement of Tryptophan and Kynurenine Concentrations in PBMC Supernatants

After incubation, supernatants were harvested by centrifugation and tryptophan and kynurenine concentrations were measured by high performance liquid chromatography (HPLC) using 3-nitro-L-tyrosine as an internal standard (Widner et al., 1997, Clin. Chem. 43, 2424-2426). To estimate the activity of IDO, kyn/trp was calculated and expressed as μmol kynurenine/mmol tryptophan. No influence of ethanol (0.1% final concentration) was detected on tryptophan degradation (data not shown).

Measurement of neopterin and IFN-γ concentrations in the supernatant of PBMC In all experiments with PBMC, neopterin concentrations were measured by ELISA (BRAHMS, Hennigsdorf/Berlin, Germany). In addition, in a subgroup of 3 PBMC experiments with 2 parallels IFN-γ concentrations were determined by ELISA (R&D International, Minneapolis, Minn.). ELISAs were run according to the manufacturer's instructions.

### Quantitative Real-Time Reverse Transcription Polymerase Chain Reaction (RT-PCR)

For quantification of IDO and IFN-γ gene expression, RNA was extracted from PBMC using Trizol reagent (Invitrogen, Vienna, Austria) and reverse-transcribed using Superscript II reverse transcriptase (Invitrogen). Thirty cyles of PCR were performed using Sure Start Taq polymerase (Stratagene, La Jolla, Calif.). Levels of mRNA were quantified by real-time PCR with the ABI/PRISM 7700 sequence detection system (PE Applied Biosystems, Foster City, Calif.). 18sRNA was used as an invariant endogenous control. Specific primers and an internal fluorescent TaqMan probe were designed as follows: human 18sRNA primers, 5′-CCATTCGAACGTCTGCCCTAT-3′ (SEQ ID NO:1) and 5′-TCACCCGTGGTCACCATG-3′ (SEQ ID NO:2); 18sRNA probe, 5′-FAM-ACTTTCGATGGTAGTCGCCGTGCCT-TAMRA-3′ (SEQ ID NO:3); human IDO primers, 5′-TGGCCAGCTTCGAGAAAGA-3′ (SEQ ID NO:4) and 5′-GCGCTGTGACTTGTGGTCTGT-3′ (SEQ ID NO:5); IDO probe, 5′-FAM-AGAAGTTAAACATGCTCAGCATTGATCA-TAMRA-3′ (SEQ ID NO:6); human IFN-γ primers, 5′ACTCATCCAAGTGATGGCTGAAC-3′ (SEQ ID NO:7), 5′-CCTTGAAACAGCATCTGACTCCTT-3′ (SEQ ID NO:8); IFN-γ probe, 5′-FAM-TCGCCAGCAGCTAAAACAGGGAAGC-TAMRA-3′ (SEQ ID NO:9). Relative mRNA expression was calculated by dividing the relative quantity of each PCR product by the relative quantity of 18sRNA in each sample.

### Measurement of Cell Viability

After incubation of PBMC with mitogens Con A or PHA (each 10 μg/ml) or treatment of THP-1 cells with LPS (1 μg/ml ), with or without cannabinoids for 48 h, cell viability was measured by MTT-test (3-[4,5-dimethyldiazol-2-yl]-2,5 diphenyl tetrazolium bromide) and by trypan blue exclusion method in three experiments performed in triplicates. No toxicity could be observed with solvent (0.1% EtOH; data not shown). IC50 were calculated by the CalcuSyn software from Biosoft, Cambridge, UK, using the original concept of Chou and Talalay (Chou and Talalay, 1984, Adv. Enzyme Regul. 22, 27-55).

### Statistics

Data are represented as mean values ±S.E.M. Because not all data sets showed normal distribution, non-parametric Friedman- and Wilcoxon-test were applied for comparison of grouped data, p-values less than 0.05 were considered to indicate significant differences.

### Results

### Effect of THC and CBD on Cell Proliferation

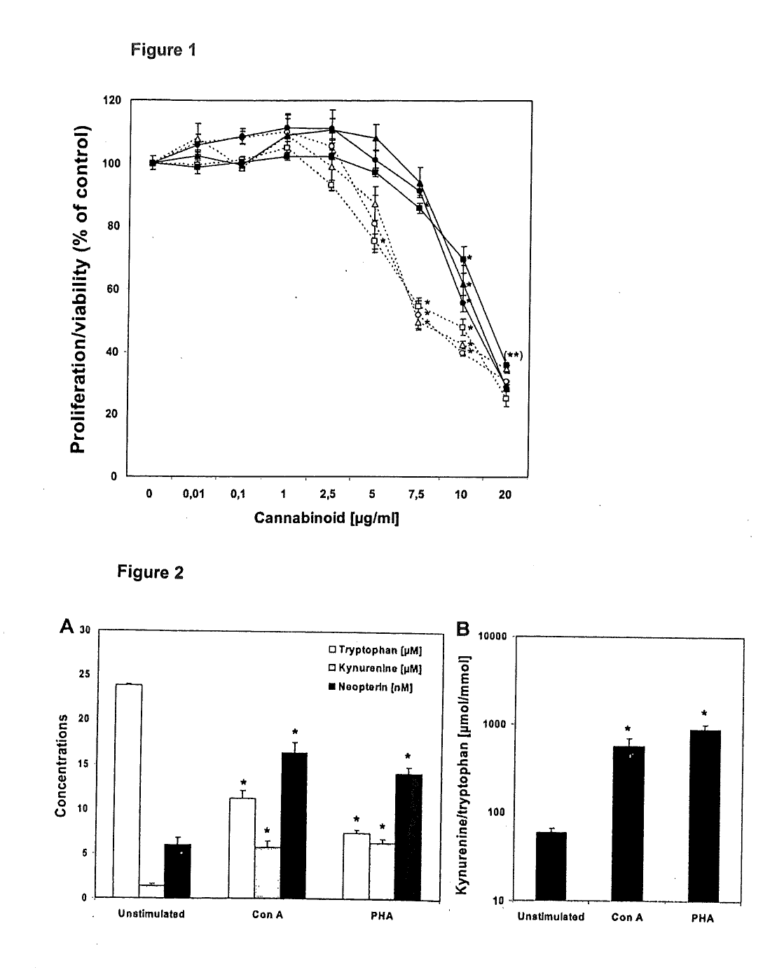

THC and CBD were evaluated for cytotoxic activity in vitro on PBMC and THP-1 cells. Treatment of PBMC with THC or CBD (0.01-20 μg/ml) dose-dependently decreased the number of viable cells in the cultures (FIG. 1), IC50 values were 14.5 μg/ml (THC) and 7.3 μg/ml (CBD) in unstimulated, 17.5 μg/ml (THC) and 8.6 μg/ml (CBD) in Con A-, and 15.2 μg/ml (THC) and 8.5 μg/ml (CBD) in PHA-stimulated PBMC. To calculate IC50 for IDO activity and neopterin formation, only results obtained at concentrations with no or minor influence on the viability of cells were used (THC≦7.5 μg/ml, CBD≦5 μg/ml).

Viability of THP-1 cells was reduced to 75-80% after treatment with LPS (1 μg/ml) for 48 hours, and co-incubation with THC or CBD (0.1-10 μg/ml) did not show any influence on the viability of unstimulated or LPS stimulated THP-1 cells (data not shown).

### Effect of THC and CBD on Tryptophan Metabolism and Neopterin Formation in Unstimulated PBMC

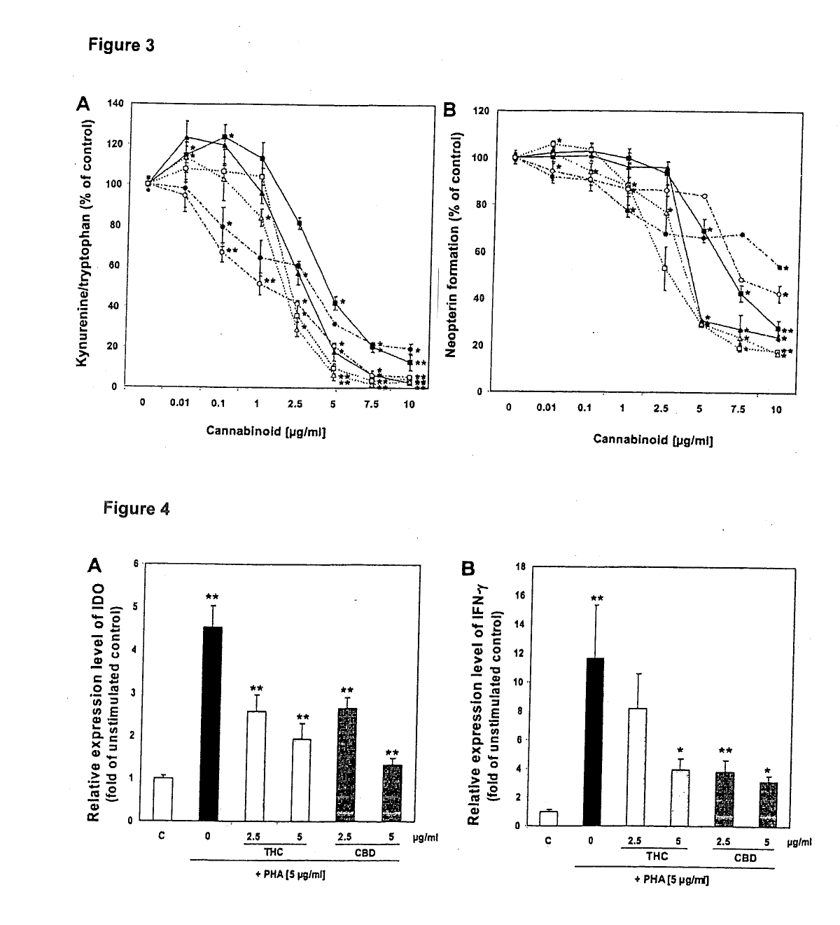

The supernatants of unstimulated PBMC contained an average concentration of 23.8±0.1 μM tryptophan and 1.4±0.1 μM kynurenine resulting in kyn/trp of 59±7.1 μmol/mmol (FIGS. 2A and B). Treatment of unstimulated cells with THC or CBD (0.01-10 μg/ml) led to a concentration-dependent decrease of kyn/trp with an IC₅₀ of 2.6 μg/ml for THC and 1.2 μg/ml for CBD, respectively (FIG. 2A). Within the same experiments, an average concentration of neopterin of 6.0±0.8 nM was detected in the supernatants of unstimulated PBMC (FIG. 2A). Also, the release of neopterin after treatment with cannabinoids was suppressed in a dose-dependent manner (IC₅₀ for THC: 16.7 μg/ml and for CBD: 8.2 μg/ml; FIG. 3B).

### Effect of THC and CBD on Tryptophan Metabolism and Neopterin Formation in Mitogen-Stimulated PBMC

In PBMC stimulated with mitogens Con A or PHA, a significant decrease of tryptophan concentrations (11.2±0.8 μM and 7.4±0.4 μM, respectively) and a concurrent increase of kynurenine concentrations (5.8±0.6 μM and 6.3±0.4 μM, respectively) were detected in the supernatants (FIG. 2A). Activation of IDO was indicated by an increase of kyn/trp, about 9-fold in Con A-treated and about 15-fold in PHA-treated cultures as compared to unstimulated cells (FIG. 2B).

Whereas low doses of THC or CBD (0.01-0.1 μg/ml) induced a modest but significant increase in the activity of the IDO enzyme, treatment of cells with high doses of cannabinoids suppressed mitogen-induced IDO activity significantly (FIG. 3A), IC50 in Con A-stimulated cells 4.2 μg/ml for THC and 2.8 μg/ml for CBD, and in PHA-stimulated cells 5.5 μg/ml for THC and 1.4 μg/ml for CBD.

Parallel measurements of neopterin concentrations in the PBMC supernatants revealed an increase of up to 16.4±1.2 nM in Con A- and to 14.2±0.6 nM upon PHA-stimulation (FIG. 2A). Co-treatment of PBMC with cannabinoids efficiently counteracted the mitogen-induced neopterin production with IC50 of 6.7 μg/ml for THC and 3.1 μg/ml for CBD in Con A-, and 5.3 μg/ml for THC and 3.7 μg/ml for CBD in PHA-stimulated cells (FIG. 3B).

### Effect of THC and CBD on IFN-γ Secretion in PHA Stimulated PBMC

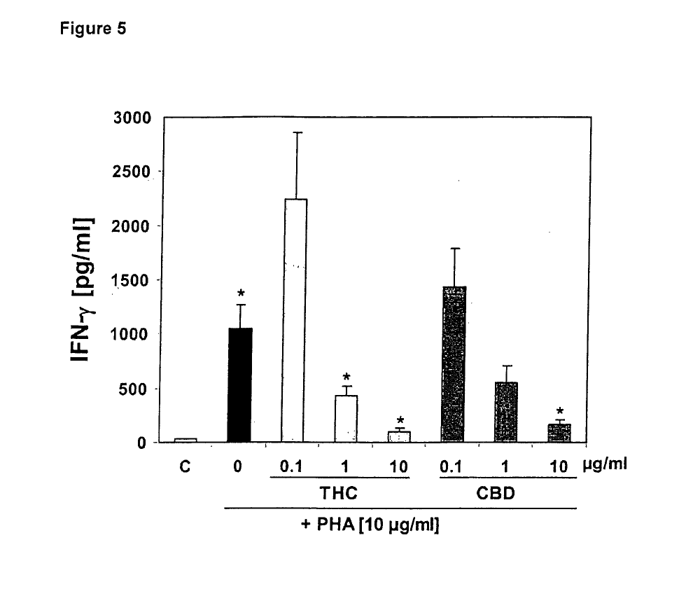

The amount of IFN-γ released into the supernatant of PBMC increased upon stimulation with 10 μg/ml PHA. Co-treatment with cannabinoids for 48 h showed a moderate but significant increase of IFN-γ secretion at low dose (0.1 μg/ml) whereas at higher doses (1-10 μg/ml) IFN-γ secretion was significantly suppressed (FIG. 5).

Effect of THC and CBD on mRNA Levels of IDO and IFN-γ in PHA Stimulated PBMC

TaqMan gene expression analyses showed that both cannabinoids exert an inhibitory capacity on the induction of IDO and IFN-γ mRNA. Stimulation of PBMC with 5 μg/ml PHA induced an about 4-5 fold increase of IDO mRNA (FIG. 4A) and a 9-15 fold increase of IFN-γ mRNA-levels after 6 h (FIG. 4B). Co-treatment of cells with THC or CBD revealed that both cannabinoids efficiently, and almost completely, inhibit mitogen-stimulated expression of IDO and IFN-γ at the highest concentration tested (5 μg/ml; FIGS. 4A and B).

### Effect of THC and CBD on Tryptophan Metabolism in LPS Induced THP-1 Cells

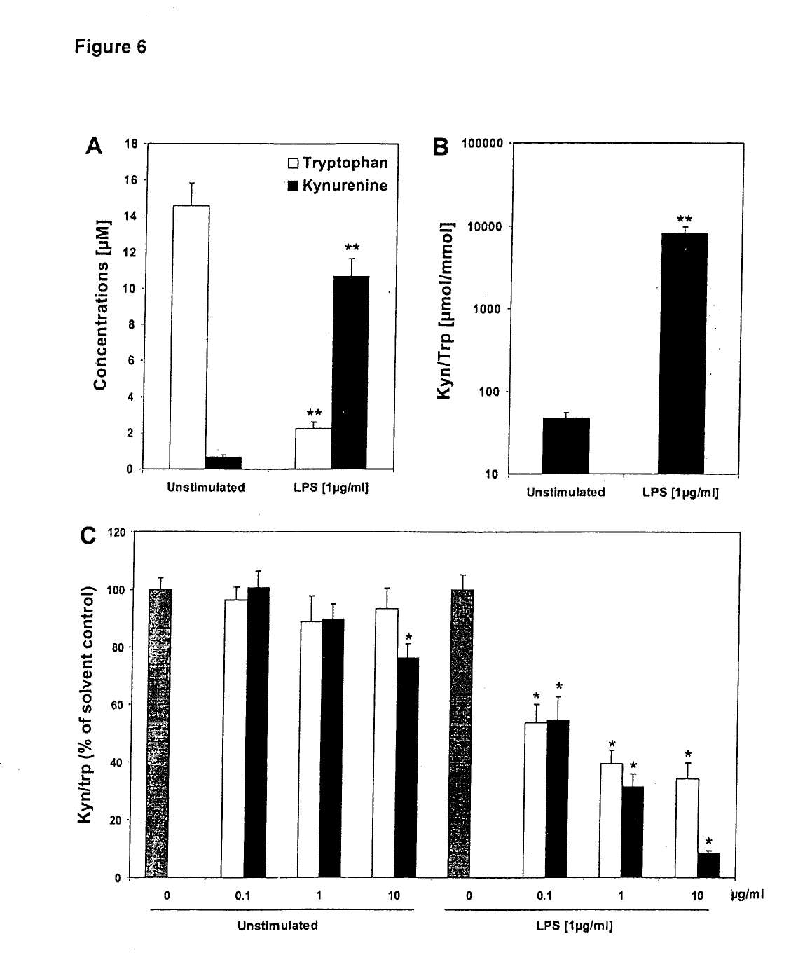

Supernatants of unstimulated THP-1 cells contained 14.6±1.3 μM tryptophan and 0.7±0.1 μM kynurenine, kyn/trp was 48±7.3 μmol/mmol (FIGS. 5A and B). Treatment of unstimulated THP-1 cells with THC did not influence tryptophan metabolism, whereas CBD showed a faint suppression of tryptophan degradation at the highest concentration tested (FIG. 6C). Stimulation of THP-1 cells with 1 μg/ml LPS lowered tryptophan to 2.3±0.4 μM which was accompanied by an increase of kynurenine concentrations to 10.7±1.0 μM (FIG. 5A), kyn/trp=8098±1608 μmol/mmol (FIG. 6B). Co-treatment of THP-1 cells with THC or CBD suppressed LPS-induced tryptophan degradation efficiently, indicated by a decrease of kyn/trp with an IC50 of 0.6 μg/ml (THC) and 0.3 μg/ml (CBD; FIG. 6C). No influence of ethanol (0.1% final concentration) was detected on tryptophan degradation (data not shown).

### Discussion

Since therapeutic applications of THC are limited by its psychoactive properties, non-psychotropic CBD, with analogue anti-inflammatory activities, has attracted interest and is also in the focus of this study, in which we investigated the potential of THC and CBD to modulate cell-mediated (Th1-type) immune response in human PBMC and in myelomonocytic THP-1 cells in vitro.

Both cannabinoids suppressed proliferation of unstimulated and of mitogen-stimulated PBMC, CBD was effective at about half the concentration as compared with of THC. In contrast, viability of THP-1 monocytes was not affected by the tested cannabinoids at doses of up to 10 μg/ml.

In PBMC, both cannabinoids efficiently suppressed mitogen-induced tryptophan degradation in a dose-dependent manner with IC50 in the low micromolar concentration range in unstimulated and mitogen-stimulated PBMC. Comparing the suppression of mitogen-induced tryptophan degradation, CBD is about 2 times more active than THC to interfere with IDO activity. Within the concentration range of 2.5 -5 μg/ml, both cannabinoids exerted an inhibitory effect also on the expression level of PHA-induced IDO mRNA. Neopterin formation was also diminished in a concentration-dependent manner. Again, CBD had an about 2-fold stronger capacity to suppress mitogen-induced neopterin formation than THC. The inhibition of tryptophan degradation and neopterin formation in parallel, suggests a suppressive effect of THC and CBD on activated T-cells and on the production of IFN-γ which could be confirmed in PHA induced PBMC on the level of IFN-γ mRNA expression as well as on the level of IFN-γ secretion. Due to the high concentrations needed to measure an inhibitory effect (1-5 μg/ml) on the biochemical pathways investigated, we assume that these effects do not depend on activation of cannabinoid receptors, but are rather mediated by direct membrane interactions based on the highly lipophilic properties of the tested cannabinoids.

Interestingly, low doses of THC and CBD (0.01-0.1 μg/ml) induced a moderate enhancement of mitogen-induced IFN-γ secretion which is well in line with a significant enhancement of IDO activity observed within this concentration range. Although the majority of available literature shows inhibitory capacities of cannabinoids on cells of the immune system, there are also reports demonstrating stimulatory activities. THC and CBD were both shown to decrease TNF-γ production in human NK cells and PBMC, respectively, whereas THC was also demonstrated to increase TNF-γ production in human monocytes. Similarly, lower doses of these cannabinoids, comparable to plasma levels found after smoking marijuana (10-100 ng/ml), was demonstrated to stimulate IFN-γ formation, whereas IFN-γ production was found to be suppressed in human PBMC at higher concentrations of THC or CBD (5-20 μg/ml). These contradictory findings result in a biphasic response relative to the cannabinoid ligand concentration applied, because most of reports showing stimulatory capacities were reported at lower doses, in the nanomolar concentration range, whereas inhibitory activities of cannabinoids were found in the micromolar concentration range. In this regard, cannabinoids were demonstrated to inhibit or to induce Th1- as well as Th2-type cytokines. Our results, demonstrating an enhancement of mitogen-induced IDO activity and secretion of IFN-γ at concentrations of 10-100 ng/ml and suppression at higher doses (1-10 μg/ml), further confirm these findings.

The suppression of IFN-γ production and of biochemical pathways related to it in PBMC document an effect of cannabinoids on T-cell stimulation. However, investigations of LPS-induced THP-1 cells show that THC and CBD also suppress tryptophan degradation directly in monocytic cells. The inhibition of IDO enzyme activity (kyn/trp) was achieved at even lower concentrations of cannabinoids than necessary in PBMC. Thus, cannabinoids have the ability to suppress tryptophan degradation mediated by T cell-derived IFN-γ but also directly in stimulated monocytic cells.

Our findings are in vitro only, however, they may have manyfold consequences also for the in vivo situation. In patients, significant correlations were found between blood levels of IFN-γ, neopterin and kyn/trp in various diseases such as human immunodeficiency virus infection, malignancy and autoimmune syndromes. Moreover, significant associations exist between the decrease of tryptophan levels and the increased susceptibility of patients for mood disturbances and depression. Activation of IDO could represent a link between the immunological network and the pathogenesis of depression, when the availability of tryptophan limits serotonin biosynthesis.

Cannabis causes very complex subjective experiences in humans such as mood elevation, enhanced sensitivity to external stimuli, and relaxation (American Psychiatric Association, 1994). Among recreational users, Cannabis is commonly accepted to possess the capacity to improve mood, lift spirits and make people feel good. The involvement of the endocannabinoid system in mood regulation and depression is very complex and consistently controversial discussed. Behavioral studies revealed on the one hand an antidepressant effect of CB1 receptor antagonists in the mouse behavioral assay, whereas on the other hand an antidepressant effect was reported by others with CB1 receptor agonists in the forced swim assay of rodents. In the rat hippocampus, THC treatment was reported to reduce hydroxytryptamine (5-HT; serotonin) turnover and the CB1 receptor antagonist SR141716 was shown to stimulate serotonin release from the prefrontal cortex. In contrast, experiments analyzing the content of serotonin in different brain regions of adult rats revealed, a marked increase of serotonin in the frontal cortex of rats chronically treated with THC. Furthermore the implication of the endocannabinoid system in depression has been linked to the serotonergic system via the activation of serotonin receptors 5-HT₁, 5-HT₂, 5-HT₃, or the enhanced firing activity of serotonergic and noradrenergic neurons after treatment with URB597, an inhibitor of the endocannabinoid hydrolyzing enzyme fatty acid amid hydroxylase (FAAH). Although clinical trials of cannabinoids in affective disorders have yielded mixed results, many patients continue to report benefits from its use in primary or secondary depressive syndromes.

Our results concerning the strong suppressive effect of THC and CBD on activation-induced tryptophan degradation and thus the activity of IDO, indicates an ability of these cannabinoids to modulate the serotonergic system, when THC or CBD may lead to an increase of tryptophan in the circulation. Aside from its role as a protein-component, the essential amino acid tryptophan is a precursor for the biosynthesis of the neurotransmitter serotonin, which is strongly involved in the pathogenesis of mood disorders and depression.

In summary, this study shows inhibition of pro-inflammatory cascades by cannabinoids Δ9-tetrahydrocannabinol (THC; Dronabinol) and cannabidiol (CBD) including the down-regulation of tryptophan degrading enzyme indoleamine (2,3)-dioxygenase (IDO). According to these in vitro results, CBD is about two times more effective than THC to suppress mitogen-induced tryptophan degradation, neopterin formation and production of interferon-γ in stimulated human peripheral blood mononuclear cells. Cannabinoids, and in particular CBD, effectively inhibited tryptophan degradation also in lipopolysaccharide-stimulated myelomonocytic THP-1 cells. Thus, the anti-inflammatory activity of CBD is achieved via suppression of T-cell activation and interferon-γ production but also by a direct influence on monocytes.

Accelerated tryptophan degradation was described in patients suffering from inflammatory conditions such as infections and malignancies. The thereby lowered circulating tryptophan levels are related to a greater risk of depression and cognitive impairment. This is most probably related to the fact that the essential amino acid tryptophan is a precursor of the neurotransmitter 5-hydroxytryptamin (serotonin) which is considered to be strongly involved in the pathogenesis of mood changes and in cognition. It can be assumed that any suppressive effect of specific cannabinoids on the degradation of tryptophan by IDO might enhance the availability of tryptophan for serotonin biosynthesis. CBD, better than THC, may increase the availability of tryptophan in vivo and may thus accelerate the biosynthesis of serotonin, and in turn improve quality of life and cognition.

Earlier we have already observed that cognitive impairment of patients with HIV infection correlates with diminished serum tryptophan concentrations and a concomitant increase of neopterin levels. Likewise in patients with various forms of cognitive impairment and dementia including Alzheimer's disease, vascular dementia and Chorea Huntington significantly lower tryptophan concentrations were observed, which correlated with the degree of cognitive impairment and with the survival of patients. These observations allow to state that counteracting tryptophan depletion might be able to slow down the processes which are deeply involved in the pathogenesis of various forms of dementia.

FIG. 1: Proliferation/viability evaluated by MTT-assay, expressed as % of control in unstimulated (circles), concanavalin A (Con A; squares)- or phytohemagglutinin (PHA; triangles)-stimulated PBMC (each 10 μg/ml) in the absence or presence of increasing concentrations of Δ9-tetrahydrocannabinol (black symbols) or cannabidiol (white symbols). Mean values±S.E.M. are shown of three independent experiments run in duplicates (*P<0.05; **P<0.005).

FIG. 2: A Concentrations of tryptophan (white bars), kynurenine (grey bars), measured by HPLC and neopterin (black bars), measured by ELISA in the supernatant of unstimulated and concanavalin A (Con A)- or phytohemagglutinin (PHA)-stimulated PBMC (10 μg/ml each). B Indoleamine 2,3-dioxygenase (IDO) activity indicated by the kynurenine to tryptophan ratio in unstimulated and mitogen stimulated PBMC, plotted in log scale. Mean values±S.E.M. are shown of four independent experiments run in duplicates (*P<0.005).

FIG. 3: A IDO activity indicated by the kynurenine to tryptophan ratio and B concentrations of neopterin, measured by ELISA, expressed as % of control in unstimulated (circles), concanavalin A (Con A; squares)- or phytohemagglutinin (PHA; triangles)-stimulated PBMC (each 10 (g/m1) in the absence or presence of increasing concentrations of Δ9-tetrahydrocannabinol (black symbols) or cannabidiol (white symbols). Mean values±S.E.M. are shown of four independent experiments run in duplicates (*P (0.05; **P<0.005).

FIG. 4: Effects of Δ9-tetrahydrocannabinol (THC, light grey bars) and cannabidiol (CBD, dark grey bars) on mRNA expression of indoleamine 2,3-dioxygenase (IDO) (A) and interferon-γ (IFN-γ (B) shown as fold of unstimulated control. Gene expression was quantified by quantitative real-time RT-PCR in unstimulated control (C) and PHA (5 μg/ml) stimulated (filled bars) PBMC co-treated or not with cannabinoids for 6 hours. Values are relative to the gene expression of 18s rRNA. Mean values±S.E.M. are shown of five independent experiments with two parallels, each measured in triplicates (*P<0.05; **P<0.005).

FIG. 5: Effects of Δ9-tetrahydrocannabinol (THC, light grey bars) and cannabidiol (CBD, dark grey bars) on interferon-γ (IFN-γ secretion, measured by ELISA. IFN-γ concentrations were determined in the supernatant of unstimulated (C) and phytohemagglutinin (PHA; 10 μg/ml) stimulated PBMC (filled bars), co-treated or not with cannabinoids for 48 h. Mean values±S.E.M. are shown of five independent experiments run in duplicates (*P<0.05).

FIG. 6: A Concentrations of tryptophan (white bars) and kynurenine (black bars) in the supernatant of unstimulated and lipopolysaccharide (LPS; 1 μg/ml) stimulated THP-1 cells, measured by HPLC. B IDO activity indicated by the kynurenine to tryptophan ratio (kyn/trp) in unstimulated and lipopolysaccharide (LPS; 1 μg/ml) stimulated THP-1 cells, plotted in log scale. C IDO activity indicated by the kynurenine to tryptophan ratio (kyn/trp) expressed as % of control (grey bars) in unstimulated and LPS (1 μg/ml) stimulated THP-1 cells in the absence or presence of increasing concentrations of Δ9-tetrahydrocannabinol (white bars) or cannabidiol (black bars). Mean values±S.E.M. are shown of three independent experiments run in duplicates (*P<0.05).

1. A method for the treatment of a disease or prevention of a condition benefiting from a reduced activity of the enzyme indoleamine 2,3-dioxygenase (IDO), the method comprising:
preparing a dietary supplement or medicament comprising at least one cannabinoid, and
administering to a human patient in need thereof an amount of said dietary supplement or medicament sufficient to reduce activity of the enzyme indoleamine 2,3-dioxygenase (IDO) for a period sufficient to treat or prevent said disease or condition.

2. A dietary supplement and/or medicament for preventing a disease or condition benefiting from a reduced activity of the enzyme IDO, wherein the dietary supplement or medicament comprises at least one cannabinoid, wherein the ratio THC:CBD (w/w) is from 1:20 to 1:1000.

3. The method according to claim 1, wherein the at least one cannabinoid is a modulator, and is administered in a dosage which leads to an increased level of circulating tryptophan.

4. The method according to claim 1, wherein the at least one cannabinoid is Δ9-tetrahydrocannabinol (THC) or cannabidiol (CBD) or a derivative thereof or a combination of THC and CBD or derivatives thereof.

5. The method according to claim 4, wherein the at least one cannabinoid is a combination of THC and CBD or derivatives thereof or a mixture of cannabinoids comprising THC and CBD or derivatives thereof.

6. The method according to claim 5, wherein the mixture of cannabinoids has less than 10% and/or more than 15% w/w CBD.

7. The method according to claim 4, wherein the at least one cannabinoid is CBD or a derivative thereof.

8. A method for the treatment or prevention of cognitive impairment or dementia, the method comprising:
diagnosing a human patient as having cognitive impairment or dementia possibly attributable to low tryptophan for serotonin biosynthesis,
preparing a dietary supplement or medicament comprising at least one cannabinoid, and
administering to said diagnosed human patient an amount of said dietary supplement or medicament sufficient to reduce activity of the enzyme indoleamine 2,3-dioxygenase (IDO) for a period sufficient to treat or prevent said disease or condition.

9. The method according to claim 8, wherein the disease or condition is selected from the group consisting of vascular dementia, Lewy body dementia, frontotemporal dementia, HIV-associated dementia, dementia pugilistica, corticobasal degeneration or hereditary dementia.

10. The method according to claim 8, wherein the disease or condition is selected from the group consisting of dementia being involved with a basic and underlying disease such Huntington, Parkinson, Alzheimer or Creutzfeldt-Jakob disease.

11. The method according to claim 1, wherein the at least one cannabinoid is derived from plant extracts or an extract comprising at least one cannabinoid.

12. The method according to claim 11, wherein the extract has less than 10% w/w THC and/or more than 15% w/w CBD.

13. The method according to claim 11, wherein the extract is substantially free of THC or the content of THC is 0% w/w cannabinoids and the main component of at least one cannabinoid is CBD.

14. The method according to claim 5, wherein the mixture of cannabinoids has less than 5% w/w THC and/or more than 20% w/w CBD.

15. The method according to claim 5, wherein the mixture of cannabinoids has less than 2% w/w THC and/or more than 25% w/w CBD.

16. The method of claim 8, wherein said dietary supplement or medicament sufficient is administered in daily doses of 20 to 1000 mg of active substance per 70 kg of body weight.

17. The method of claim 8, wherein said dietary supplement or medicament is administered in daily doses sufficient to register levels in the brain of 1-10 μg/ml.

18. The method according to claim 12, wherein the mixture of cannabinoids has less than 5% w/w THC and/or more than 20% w/w CBD.

19. The method according to claim 12, wherein the mixture of cannabinoids has less than 2% w/w THC and/or more than 25% w/w CBD.
